(19) United States
(12) Patent Application Publication
(10) Pub. No.: US 2011/0123654 A1
(43) Pub. Date: May 26, 2011

(54) SYNERGISTIC ENHANCEMENT OF CELLULAR ERGOGENIC NUTRIENT UPTAKE, LIKE CREATINE OR CARNITINE, WITH TARRAGON

(76) Inventors: Ralf Jaeger, Sankt Augustin (DE); Martin Purpura, Bonn (DE); Ivo Pischel, Rossbach (DE)

(21) Appl. No.: 12/996,163

(22) PCT Filed: Jun. 5, 2009
(86) PCT No.: PCT/EP09/56921

§ 371 Date: Jan. 31, 2011

Related U.S. Application Data
(60) Provisional application No. 61/131,139, filed on Jun. 6, 2008.

(30) Foreign Application Priority Data
Jun. 6, 2008 (EP) 08157772.8

(51) Int. Cl. A61K 36/282; A61K 38/02; A61K 31/70; A61K 31/205; A61P 25/00; A61P 3/10; A61P 3/04; A61P 3/00

(52) U.S. Cl. 424/740; 514/1.1; 514/23; 514/556

(57) ABSTRACT

A product comprising a) Russian Tarragon (Artemisia dracunculus), an extract of Russian Tarragon or a derivative thereof b) a creatine and/or carnitine compound or derivative or precursor thereof.

The present invention relates to combinations of creatine and/or carnitine components with further compounds, the preparation and their use.

The modern civilization especially of the western world is threatened by a number of diseases related to the rising prosperity. Thus, the incidences of obesity, cardio-vascular and metabolic diseases, like metabolic syndrome, increased blood lipids and diabetes type II as well as neurodegeneration (Alzheimers) are increasing dramatically. Especially, the ingestion of high amounts of simple sugars may lead to high blood concentrations of glucose and thus, after a longer period of time, to glucose intolerance, metabolic syndrome and often finally to diabetes type II. Very critical in this context are the high glucose peaks that occur post-prandial, which can lead to glycoxylation reactions in the blood and certain tissues, probably followed by secondary diseases.

Additionally, the lack of physical workout and intake of diets characterized by high fat intake and repeated ingestion of refined foods and sugars, coupled with low fiber and vegetable intake, along with the natural aging process, causes a deterioration in the way in which the body metabolizes blood glucose. When the body cannot properly metabolize blood glucose, a tendency to store glucose as fat typically occurs. This is one reason levels of body fat increase with age. On the other hand constantly high blood concentrations of glucose or high post-prandial glucose peaks can be related to undesirable glycoxylation reactions of blood or tissue components. Secondarily, a glucose tolerance and/or insulin resistance can be developed, which may be related to metabolic syndrome and diabetes type 2. Diabetes is also known to be associated with a variety of other ailments including heart disease, hypertension, and obesity. There is a known link between insulin resistance and increased visceral adiposity. Diabetes is also a leading cause of glaucoma and other conditions related to a decrease in the quality of life.

While aging a loss of muscular mass and power as well a general deterioration of health, physical performance, mobility and cell viability takes place and the incidence of metabolic diseases, for instance diabetes as well as the fall of mental acuity occurs. Comparable condition may be found in some cases of ill-health situations.

The object of this invention was thus to develop a product and a method for enhancing the cellular uptake of creatine and/or carnitine nutrients, especially into certain cellular tissues to increase their cellular bioenergetical status while lowering high blood glucose levels in the blood. Therefore, the reason for this invention is to prevent or counteract (treat) the general deterioration of the health status while aging or to increase the bioenergetic level of certain tissue, especially under the condition of illnesses and/or during sports.

In general there exists a correlation between the cellular concentration of ergogenic nutrients of certain cells and the resistance and viability of these cells against stress factors. In general, the higher the concentration is the higher the cellular survival rate, and thus a apoptosis of the cells can be reduced. These mechanisms are extremely important for the very sensitive cell of the neuronal tissue and nerve cells.

### The Object is Solved by a Product Comprising

a) Russian Tarragon (Artemisia dracunculus), an extract of Russian Tarragon or a derivative thereof
b) a creatine and/or carnitine compound or derivative or precursor thereof.

According to the invention, the application of the disclosed combination of a Russian tarragon extract and an ergogenic compound synergetically increases the uptake of nutritive glucose and creatine and/or carnitine nutrients, like creatine from the blood to the said tissues, like skeletal and very likely neuronal cells.

Surprisingly, it was found that the oral administration of disclosed combination of Russian tarragon extract and a creatine compound leads to a change of creatine blood levels, which are due to the supposed increase of creatine uptake to the said tissues and therefore to the increased cellular bioenergetical level. Due to the fact that the disclosed method of use results in a synergetically increases the uptake of nutritive glucose and creatine and/or carnitine nutrients, e.g. creatine from the blood to the said tissues, like skeletal and very likely neuronal cells, this method has a supra-additive effect, which was not foreseeable.

Disclosed herein are inter alia orally applicable combinations comprising an water-soluble extract of Russian Tarragon, and a form of a creatine compound, like creatine monohydrate, salts or derivatives of creatine or mixtures thereof and a nutrient, or a derivative or a precursor thereof without or with a carbohydrate and a method of increasing the absorption of nutrients into mammalian tissue, therefore enhancing nutrient transport, and athletic performance comprising administration of the combination as dietary supplement, food or drink preparation, or in a pharmaceutical delivery form.

Accordingly, an embodiment of the invention is a method and a combination which will enhance the absorption of nutrients into skeletal muscles or nerve cells. More specifically, to provide a method and a combination as dietary supplement that will enhance the absorption of creatine into skeletal muscle and nerve cell.

It is further an embodiment of the invention to stimulate an insulin dependant pathway for absorption of creatine into the skeletal muscle.

Furthermore, it is an embodiment of this invention to provide a method and an orally applicable combination which will avoid fatigue promote strength, stamina and higher performance during sports activities and/or under illness conditions as well as weight loss and body fat reduction.

Creatine is a natural dietary component primarily found in animal products, especially in meat and meat products. In the body, creatine is stored predominantly in skeletal muscle, and mostly in the form of phosphorylated creatine, called phoshocreatine and creatine phosphate, but can be found also in its free form. Total creatine content of mammalian skeletal muscle (i.e., creatine and phosphorylated creatine) typically varies from about 100 to about 140 mmol/kg. The level of creatine and phosphorylated creatine present in skeletal muscle can be increased through dietary supplementation with creatine, its monohydrate, salts or other derivates, like esters or its metabolic precursors.

The biochemical fuel for all muscular or bioenergetic work in the body is adenosine tri-phosphate, or ATP. During intense exercise, ATP is utilized very rapidly. The body does not store much ATP in muscle so other substances must be broken down in order to replenish the ATP that is rapidly declining during exercise. If the ATP is not replenished, fatigue occurs and force/power production decreases. Of all the substances in the body that can replenish ATP, the fastest is phosphocreatine. Thus, the primary function of phosphocreatine in muscle is to buffer ATP by preventing decreases in ATP during exercise. ATP and phoshocreatine play an important role in the bioenergetic metabolism of neuronal tissue and nerves, too.

Creatine has long been known to increase cellular energy due to its involvement in metabolic cell processes. Thus, creatine plays a role in many health and disease conditions (Creatine and Creatine Kinase in Health and Disease, Springer, Heidelberg, Germany, Ed. G S Salomons, M Wyss, 2007). Especially, in the muscle and nerve cells creatine and its phosphorylated form as Phosphocreatine play a crucial role as an energy buffer for the transphosphorylation reaction towards adenosine triphosphate (ATP) as universal currency for cellular energy. Therefore, creatine and its derivates has been use as sports supplement (Creatine: A Review of Efficacy and Safety; Graham A S, Hatton R C, J Am Pharm Assoc 39(6):803-810, 1999) and for improving neuro-muscular diseases.

Creatine is taken up into tissues, such as skeletal muscle and neuronal tissues and nerves, by means of an active transport system that typical involves an insulin dependent pathway, especially in the case of skeletal muscles. In a study by Stengee et al., insulin was co-infused along with creatine supplementation. (Am. J. Physiol., 1998; 275:E974-79). The results of this study indicated that insulin can enhance creatine accumulation in muscle, but only if insulin levels are present at extremely high or over-physiological concentrations. Stengee et al. refers to a previous study by Green et al. which involved experimentation with ingestion of creatine in combination with a carbohydrate-containing solution to increase muscular uptake of creatine by creating physiologically high plasma insulin concentrations. Stengee et al. reports that Green et al. had found the quantity of carbohydrate necessary to produce a significant increase in creatine uptake, as compared to creatine supplementation alone, was almost not acceptable for ingestion.

Thus, there exists a need in the art for a viable method of increasing the uptake of creatine into mammalian tissue, such as skeletal muscle and nerve cells. Further, there exists a need in the art for a dietary supplement whose administration at normal physiological concentrations would effect such an increase in creatine uptake.

Creatine is commercially readily available in form of its monohydrate, although in some products impurities were found, which may have side effects. The syntheses and preparation of most of the above mentioned derivatives are disclosed in different patents. Thus, Creatine Pyruvate can be prepared according to U.S. Pat. No. 6,172,111 and U.S. Pat. No. 6,166,249, Creatine Ascorbate according to U.S. Pat. No. 5,863,939, Creatine citrates and stabilized Creatine citrates according to U.S. Pat. No. 5,973,199 and US patent application 2005-0037069 as well as Creatine pyroglutamate according to U.S. Pat. No. 7,329,763 and Creatine ketoglutarate according to U.S. Pat. No. 7,301,051 and the like.

The administration of metabolic pre-cursors has got same purpose and will results in an increase a cellular creatine concentration.

Similar to creatine, recent research has shown that carnitine behaves in the same manner as creatine as a nutritive component and its uptake can be stimulated alike creatine.

Carnitine is a critical nutrient for normal skeletal muscle bioenergetics. Carnitine has a dual role as it is necessary for the metabolic oxidation of long-chain fatty acid, and also shuttles accumulated acyl groups out of the mitochondria. The optimization of both of these metabolic processes during peak exercise performance is required by muscles. Based on the metabolic importance of carnitine, its supplementation can be beneficial and therefore it is recommended to avoid a carnitine limitation for either fatty acid oxidation or the removal of acyl-CoAs during exercise.

Unfortunately, most of the anticipated metabolic effects of carnitine supplementation have not been observed in healthy persons, due to the rather low uptake to the said issues, e.g. muscles and nerves. The failure to demonstrate clinical efficacy of carnitine may reflect the complex pharmacokinetics and pharmacodynamics of carnitine supplementation, the challenges of clinical trial design for performance endpoints, or the adequacy of endogenous carnitine content to meet even extreme metabolic demands in the healthy state.

The combination of Russian tarragon and carnitine may overcome the low uptake of carnitine to certain tissues and cells, like skeletal muscle and nerves.

Natural and/or synthetic substances are known that may control blood glucose and enhance nutrient transport. Various modes of actions are described for such substances. For instance, some substances act by mimicking the effects of insulin and thus are able to replace endogenous insulin partly. Such substances include naturally occurring compounds, well defined chemical entities like taurine, 4-hydroxyisoleucine, and arginine, as well as trace elements like vanadium or chromium. Some of these compounds are isolated from plants, thus 4-hydroxyisoleucine was found in the Ayurvedic plant Fenugreek and may be the active principle for this plant as anti-diabetic botanical. Although these defined chemicals have been shown to act as insulinomimetics by decreasing serum blood glucose levels mostly in animal models, they have been only seldom successfully developed into appropriate treatments for glucose metabolism disorders.

Other compounds act directly to increase the so-called insulin sensitivity or glucose tolerance. Glucose intolerance forces the body to generate additional insulin in an effort to lower blood glucose. This causes stress on the beta-cells of the pancreas and is thought to be a key contributor to Type II diabetes. In a state of glucose intolerance, the body mechanism for disposing of blood glucose is not functioning at its optimum level and therefore the system is inefficient.

Substances which increase insulin sensitivity or glucose tolerance by assisting the body in returning to optimal levels of blood glucose include alpha-lipoic acid, pinitol and myo-inositol. These substances cannot entirely replace the function of endogenous insulin, but work at the receptor level alongside endogenous insulin to increase insulin sensitivity or glucose tolerance. Here, the action is exerted directly on the Glut-4 receptor of the cell to trigger the cascade normally caused by insulin that allows for the reduction in blood sugar via the transport of nutrients into the cell.

Botanicals helpful for enhancing glucose uptake into certain organ tissues are known as well. They include for instance, bitter melon (Momordica charantia), Fenugreek (Trigonella foenum-graecum), Panax ginseng, Grape Seeds, Cinnamon and many more. Phytotherapies and their combinations demonstrate multiple beneficial antidiabetic mechanisms, including modulation of carbohydrate metabolism, restoration of b-cell integrity and function, insulin-releasing activity, improvements in glucose uptake/utilization, antioxidant properties and a reduction in the risk of cardiovascular disease.

Recent papers and patent applications hypothesize that certain sub-fractions of the botanical extracts are responsible for the lowering of blood glucose levels, e.g. anthocyanins and anthocyanidins (Insulin Secretion by Bioactive Anthocyanins and Anthocyanidins Present in Fruits; Jayaprakasam B, Vareed SK, Olson L K, Nair M G; J. Agric. Food Chem., 53 (1), 28-31, 2005) and US patent application 2008-0063689)

In general terms, nutrient transport enriches the deposit of nutrients in various tissues. For instance, after the insulin cascade, the Glut-4 transport system triggered by insulin drives nutrients such as carbohydrates, amino acids including creatine into skeletal tissue.

Russian Tarragon (Artemisia dracunculus) including its varieties (e.g. var. inodora) and cultivars are the fertile, non-aromatic and bitter-tasty botanical species, which are distinguished from the so-called French or German Tarragon often used for culinary purposes. Nevertheless, Russian Tarragon (Artemisia dracunculus) and plant extracts of Russian tarragon are considered as food and generally recognized as safe (GRAS) and can be administered orally to humans or animals for the purpose of controlling blood glucose as well as improving glucose tolerance according to U.S. Pat. No. 6,893,627. This patent discloses an ethanolic plant extract of freeze-dried fresh plants derived from hydroponically grown Tarragon. Prior to this patent publication several scientific papers described the traditional use of not specified tarragon for diabetes type 2 (e.g. Swantson-Flatt S K, Day C, Flatt P R, Gould B J, Baily C J. Glycemic effects of traditional plants treatments for diabetes: studies in normal and streptozocin induced diabetic mice. Diabetes Res 1989; 34(2): 132-135).

Due to the fact that tarragon preparations can influence the glucose metabolism in an insulinomimetic manner it helps to clear glucose from the blood, reduces post-prandial glucose peak and helps reducing undesired glycoxylation reaction with vital molecular body structures or metabolites in the blood serum or tissues.

For none of the main cultivars or varieties of Russian Tarragon, the uptake of nutrients, like creatine or carnitine was demonstrated before.

The different botanical extracts were obtained by standard laboratory methods but were additionally transferable and could be up-scaled to technical production. Russian Tarragon can be used as raw material for an extraction with water or hydroethanolic extraction solvent. The raw materials were cultivated on open fields, harvested and gently dried in a conventional dried commonly used for drying herbs and spices.

Suitable extracts can for example be produced as follows: 1 kg of the raw materials is extracted twice with either 8 L water or 8 L of 20% ethanol (V/V) at 80° C. or 50° C., respectively. After cooling of the eluates over night, the solutions are filtered through paper filters and the solvent is evaporated by means of a rotatory evaporator. The obtained dense extracts were mixed with 30% of suitable carrier, like maltodextrins, hydrolysed collagen, microcrystalline cellulose or cellulose derivatives, and dried at 50° C. in a drying chamber. The dried extract is finally ground and sieved for an adjustment of the particle size. The yield of native extract is about 35% and the analysis shows water contents of less than 5% and a complete removal of the essential oil (Estragol, Methyleugenol).

The herein disclosed and described combinations of Russian tarragon extracts and nutrients creatine and/or carnitine, synergistically improve the cellular energy-level by over-additive uptake of these nutrients.

U.S. Patent Application Publication No. 2004-0224035 describes the use of cinnamon extract to increase the absorption of creatine. However, still rather high amount of carbohydrates needs to be co-administrated to obtain the desired results.

The given above problem with optimizing the effects of creatine but without the utilization of large amounts of high glycemic carbohydrates to spike insulin in order to augment the flow of creatine (and other nutrients) into the muscle cell can be achieved with the herein disclosed invention.

Therefore, the disclosed invention overcomes the problem with optimizing the cellular uptake and effects of creatine without the need to utilize large amounts of high glycemic carbohydrates to spike insulin in order to augment the flow of creatine (and other nutrients) into the muscle cell.

Similarly related, such administration can also be used for the purpose of enhancing creatine transport into excitable tissues such as skeletal muscle and neuronal tissues. The material can be administered as combinations of Russian Tarragon extracts and creatine and can be administered in a variety of product forms including capsules, tablets, powdered beverages, bars, gels or drinks.

The invention relates to the use of a combination of a plant extracts from Tarragon (Artemisia dracunculus) and certain nutrients (e.g. creatine and/or carnitine and/or their derivatives) for the enhancement of cellular nutrient uptake into muscular and nerve cells, which involves the supplementary, prophylactic or therapeutic use, in particular of creatine or its monohydrate and salts and a water-soluble extract of Russian Tarragon in preferred daily dose of 1.0 g to 20 g. The disclosed combinations of Russian tarragon extracts and creatine and/or carnitine components are not restricted to any particular form of application, which makes them all the more suitable for the different application areas.

The combination and/or formula may contain glucose and other carbohydrates as well as small molecules like pyruvic acid or creatine [and carnitine (carnosine etc)] can enhance cellular energy-level, due to their involvement with cellular energy metabolism. Ideally, besides Creatine monohydrate, Creatine Pyruvate can be used, due to the fact that this salt possess itself a higher bioavailability than other creatine components (Jager R, Harris R C, Purpura M, Francaux M. Comparison of new forms of creatine in raising plasma creatine levels. J Int Soc Sports Nutr. 2007 Nov. 12; 4:17)

Disclosed herein is: (a) a combination comprising a tarragon extract, or an extract thereof or a derivative of the extract thereof and a nutrient, like creatine or carnitine or a derivative or a precursor thereof, with or without a carbohydrate; and (b) methods of increasing the uptake of said nutrients in mammalian muscle, enhancing nutrient transport, and enhancing athletic performance comprising administration of said combination as dietary supplement.

Similarly, the herein disclosed combination comprising a tarragon extract, or an extract thereof or a derivative of the extract thereof and a nutrient, like creatine or carnitine or a derivative or a precursor thereof, with or without a carbohydrate; and (b) methods of increasing the uptake of said nutrients in nerve cells, enhancing nutrient transport, and enhancing mental performance comprising administration of said composition as dietary supplement, food or drinks preparation, or in pharmaceutical delivery form.

Accordingly, it is an object of the invention to provide a method and a composition which will enhance the uptake of nutrients into mammalian muscle and nerve cells. More specifically, it is an object of the invention to provide a method and a composition which will enhance the uptake of creatine into skeletal muscle and neuronal tissue. It is a further object of the invention to provide a method and a dietary supplement that triggers an insulin dependent pathway to enhance the uptake of creatine into skeletal muscle. It is a still further object of the invention to provide a method and a dietary supplement that achieves these objects when administered in physiologically acceptable amounts.

Also disclosed herein is: (a) a composition comprising tarragon, or a water-soluble extract thereof or a derivative of the extract thereof and a nutrient like creatine or carnitine (b) methods of losing weight and reducing body fat comprising administration of said composition.

Accordingly, it is also an object of the invention to provide a method and a dietary supplement which will promote weight loss and body fat reduction.

Other objectives, advantages and features of the invention will become apparent from the given detailed description, and from the claims.

The disclosed and described combinations of Russian tarragon and creatine and/or carnitine components synergistically improve the cellular energy-level while over-additive uptake of these nutrients occurs. The administration of those combinations can also be used for the purpose of enhancing nutrient transport for purposes of athletic performance and improvement of Body Composition-Index (BCI) while controlling bodyweight and body fat levels, and therefore improves the body composition, increases wellness and mental and physical performance during sports, in illness conditions or are circumstance of special needs.

In preferred embodiments, the Tarragon component is an extract of Russian Tarragon prepared with water or water-alcohol mixtures up to 20% of alcohol.

A derivative of Tarragon is preferably a constituent of Tarragon selected from the group of carbohydrates, proteins, peptides, and polyphenols.

Preferred nutritive compounds are creatine and carnitine and mixture thereof. Suitable form for the creatine compound is selected from creatine hydrates, creatine salts, creatine monohydrate, creatine pyruvate, creatine ascorbate, creatine citrates, creatine malate, creatine tartrate, creatine orotate, creatine pyroglutamate, creatine ketoglutarate, creatine ethyl ester salts and/or precursors of creatine, such as arginine, glycine, methionine, guanidinoacetate and mixtures of thereof.

Suitable forms of carnitine are carnitine, carnitine salts, alkanoyl-carnitine, L-carnitine, L-carnitine hydrochloride, L-carnitine fumarate, L-carnitine, L-tartrate, L-carnitine Magnesium citrate, Acetyl-L-carnitine hydrochloride, 3-lauroyl-L-carnitine hydrochloride, and carnitine creatinate or mixtures thereof.

Preferred contents of the product of the invention comprise a) an amount of 100 to 25000 mg and b) in an amount from 20 to 5000 mg.

A further embodiment of the invention is a dietary supplement, a food, a beverage or a pharmaceutical product comprising the product of the invention.

It may additional comprise carbohydrates, like dextrose, maltose, maltodextrin and trehalose, formulation aids, like dissolution enhancer, binder and other auxiliaries, minerals, like Magnesium and Calcium, trace elements, like Vanadium, Chromium, Zinc, methylxanthines, like caffeine, theobromine and theophylline, free amino acids, like taurine, glutamine, citrulline, leucine, glycine, arginine, alanine, or salts of derivatives thereof, vitamins, like vitamin A, C, E, vitamin derivatives, herbs and botanical extracts with or without glucose-modifying effect, as well as lactic acid buffering agent, like (sodium) bicarbonate, citrates, phosphates, carnosine, beta-alanine, and mixtures thereof.

Suitable application form comprises a powder, capsules, tablets, effervescent tablets, powdered beverages, bars, gels or drinks, pharmaceutical delivery systems.

The product of the invention can be use for enhancing athletical muscular performance, muscle strength, muscle mass, stamina, endurance, for improvement of physiological recovery and body-composition index (BCI), for avoidance of fatigue as well as for enhancing cognitive performance, neuronal tissue stress tolerance, for avoidance of dementia and neurodegeneration and metabolic diseases, like metabolic syndrome and diabetes type 2 in athletes, sport people or elderly people and ill patients.

Further embodiment of the invention is the use of Russian tarragon (Artemisia dracunculus), an extract of Russian Tarragon or a derivative thereof to increase uptake of creatine and/or carnitine substances.

Administration of creatine for the supplementary, prophylactic or therapeutic use to increase the cellular levels of nutrients and thereby energy can the achieved through ingestion of creatine, its monohydrate or its derivatives, like physiologically applicable salts, e.g. chloride, sulphate, phosphate, ascorbate, pyruvate, orotate, citrate, malate, etc., or in form of creatine esters, e.g. creatine ethyl ester, etc. and other derivatives.

The following examples are used to explain the invention in more details without limiting the scope.

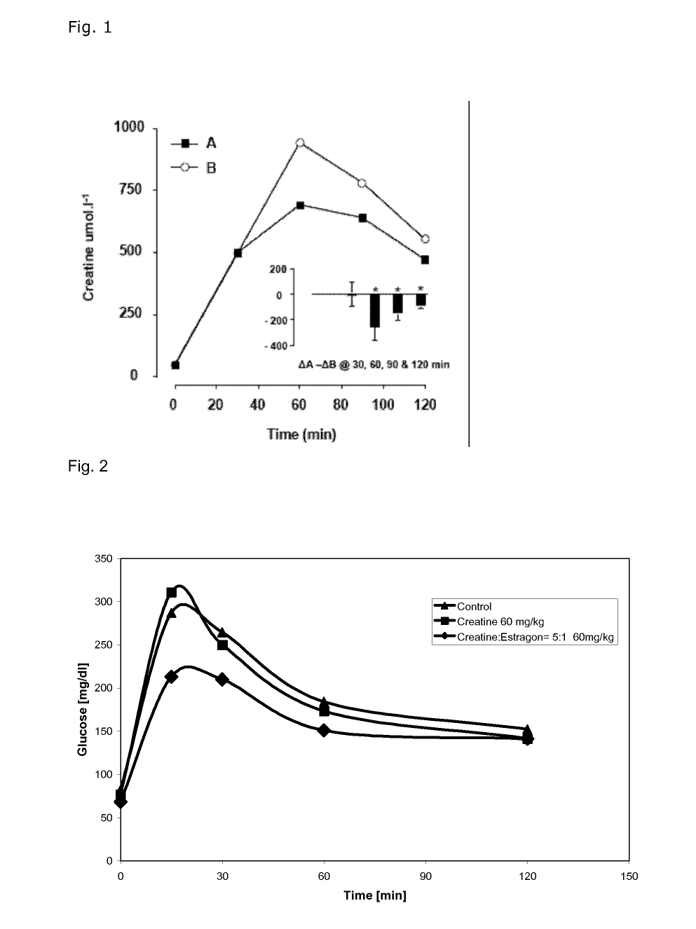

FIG. 1 shows Mean (SD) plasma creatine concentration according to example 10.

FIG. 2 shows blood glucose levels according to example 11.

### EXAMPLE 1

### Preparation of Hydroethanolic Extracts of Russian Tarragon

1 kg of the raw material of Russian tarragon was extracted twice with 8 L of 20% ethanol (V/V) at 50° C. After cooling of the eluate over night, the solutions were filtered through paper filters and the solvent evaporated by means of a rotatory evaporator. The obtained dense extracts was mixed with 30% of maltodextrin as suitable carrier, and dried at 50° C. in a drying chamber. The dried extract was finally ground and sieved for an adjustment of the particle size. The yield of native extract is about 33% and the analysis showed water contents of less than 2% and an almost complete removal of the essential oil (Estragol, Methyleugenol) of less than 3 ppm.

### EXAMPLE 2

### Preparation of Aqueous Extracts of Russian Tarragon

1 kg of the raw material of Russian tarragon was extracted twice with 8 L water at 80° C. After cooling of the eluate over night, the solutions were filtered through paper filters and the solvent evaporated by means of a rotatory evaporator. The obtained dense extracts was mixed with 30% of hydrolysed collagen as suitable carrier, and dried at 50° C. in a drying chamber. The dried extract was finally ground and sieved for an adjustment of the particle size. The yield of native extract is about 32% and the analysis showed water contents of less than 4% and a complete removal of the essential oil (Estragol, Methyleugenol).

### EXAMPLE 3

### Preparation of Tablets

| Ingredient | Amount per tablet |
|---|---|
| Russian Tarragon hydroethanolic extract | 125 mg |
| Creatine monohydrate | 400 mg |
| Creatine ascorbate | 100 mg |
| Calcium phosphate | 265 mg |
| Methylcellulose | 50 mg |
| Stearic acid | 24 mg |
| Magnesium stearate | 7 mg |
| Silicon dioxide | 10 mg |
| TOTAL | 931 mg |

The recommended daily dose is 4-6 tablets.

### Production Procedure

1. All the active substances and adjuvants are sieved through a sieve with a mesh size of 1.0 mm.
2. The tarragon extract and the excipients are weighed and introduced into a mixer. The blend is mixed for 15 minutes. The homogeneity of the mixture is checked visually.
3. The tablets are compressed directly from the mixture.

### EXAMPLE 4

### Preparation of Soft-Gelatin Capsule

Formulation:

| Ingredient | Amount per tablet |
|---|---|
| Russian Tarragon aqueous extract | 50 mg |
| Creatine monohydrate | 300 mg |
| Creatine Pyruvate | 150 mg |
| Zinc (as sulfate, chloride or pyruvate) | 15 mg |
| Medium Chain Triglycerides | 375 mg |
| TOTAL | 890 mg |

The recommended daily dose is 4-6 capsules.

Production Procedure

1. All the active substances, adjuvants, and the diluent are weighed and introduced into a mixer. The blend is mixed for 15 minutes. The homogeneity of the obtained paste is checked visually.
2. The soft-gel capsule can be filled using standard industrial equipment.

### EXAMPLE 5

### Preparation of Soft-Gelatin Capsule

Formulation:

| Ingredient | Amount per tablet |
|---|---|
| Russian Tarragon aqueous extract | 150 mg |
| L-Carnitine L-Tartrate | 250 mg |
| Acetyl-L-carnitine hydrochloride | 200 mg |
| Medium Chain Triglycerides | 375 mg |
| TOTAL | 975 mg |

The recommended daily dose is 4-6 capsules.

### Production Procedure

1. All the active substances, adjuvants, and the diluent are weighed and introduced into a mixer. The blend is mixed for 15 minutes. The homogeneity of the obtained paste is checked visually.
2. The soft-gel capsule can be filled using standard industrial equipment.

### EXAMPLE 6

### Preparation of Chewable Tablets

For the preparation of the tablets a commercial available chewable matrix based on Mannitol or Sorbitol, corn starch, sweetener, and other excipients were used.

Formulation:

| Ingredient | Amount per tablet |
|---|---|
| Russian Tarragon aqueous extract | 150 mg |
| Creatine monohydrate | 750 mg |
| Creatine Pyruvate | 250 mg |
| Chewable matrix | 1500 mg |
| Sodium bicarbonate | 50 mg |
| Flavors | 90 mg |
| Magnesium stearate | 25 mg |
| Silicon dioxide | 10 mg |
| TOTAL | 2825 mg |

The recommended daily dose is 3-4 chewable tablets.

### Production Procedure

1. All the active substances and adjuvants are sieved through a sieve with a mesh size of 1.0 mm.
2. The Tarragon extract and the excipients are weighed and introduced into a mixer. The blend is mixed for 30 minutes. The homogeneity of the mixture is checked visually.
3. The tablets are compressed directly from the mixture.

The daily dose is one chewable tablets four times a day.

### EXAMPLE 7

### Preparation of Effervescent Tablets

Formulation:

| Ingredient | Amount per tablet |
|---|---|
| Russian Tarragon aqueous extract | 150 mg |
| Creatine Pyruvate | 2500 mg |
| Sodium hydrogen carbonate | 1000 mg |
| Polyethylene glycol 4000 | 500 mg |
| Sweetener | 70 mg |
| Orange flavor | 90 mg |
| Magnesium stearate | 20 mg |
| TOTAL | 3630 mg |

The recommended daily dose is 2-4 effervescent tablets dissolved each in 300 ml of water.

### Production Procedure

1. All the active substances and adjuvants are sieved through a sieve with a mesh size of 1.0 mm.
2. The Tarragon extract, sodium hydrogen carbonate, citric acid and polyethylene glycol 4000 are weighed and introduced into a mixer. The mixture is mixed for 25 minutes. The homogeneity of the mixture is checked visually.
3. The tablets are compressed directly from the mixture.

### EXAMPLE 8

### Preparation of Nutritional Bars

Formula of the bar filling: ½ cup Sugar, 5 tb Cornstarch, 3 tb Brown sugar, ¼ ts Salt, 3 c Milk, 3 Egg yolks, beaten 1 ts Vanilla, 8 oz Chocolatebar, and 4750 mg Russian tarragon water extract, 25 grams of micronized creatine monohydrate, 1000 mg vitamin blend (e.g. Multi 10, Roche, RDA=200 mg), 1200 mg Calcium as Carbonate, 450 mg Magnesium as Carbonate, and 50 mg Zinc as Sulfate.

### Production Procedure

Combine all but vanilla and chocolate bar in a saucepan. Stir constantly until mixture boils; boil and stir 1 minute. Remove from heat; add vanilla and chocolate bar, broken into pieces. Stir until chocolate is completely melted. Add the rhodiola extracts, creatine, vitamins, and minerals. Pour into bowl and press plastic wrap directly on surface; cool. Yields about 4 cups filling or about 10 bars Of 100 grams each.

The recommended daily dose is two to four nutritional bars.

### EXAMPLE 9

### Preparation of Refreshing and Energizing Powder Drink Formulation

One serving size of this drink contains:

| | | |
|---|---|---|
| Russian Tarragon water extract | 350 | mg |
| Creatine monohydrate | 1500 | mg |
| L-Carnitine tartrate | 1000 | mg |
| Amino acids (protein hydrolysate) | 7.5 | g |
| Carbohydrates (Maltodextrin) | 7.5 | g |
| Sweetener | 70 | mg |
| Orange flavor | 90 | mg |
| Vitamin B1 | 0.7 | mg |
| Vitamin B2 | 0.8 | mg |
| Vitamin B6 | 1 | mg |
| Vitamin B12 | 0.5 | mcg |
| Vitamin C | 30 | mg |
| Vitamin E | 5 | mg |
| Niacinamide | 9 | mg |
| Folsäure | 100 | mcg |
| Biotin | 75 | mcg |
| Pantothenic acid | 3 | mg |
| Calcium | 120 | mg |
| Magnesium | 45 | mg |
| Zinc | 5 | mg |

### Production Procedure

For the production on technical scale the above shown quantities of the blend should be multi-fold with a factor up to 10000 or even higher to obtain 200 kg+batches.

1 All the active substances and adjuvants are sieved through a sieve with a mesh size of 1.0 mm.
2. The aqueous Russian Tarragon extract, creatine monohydrate and carnitine tartrate, amino acids, maltodextrin, and the other components are weighed and introduced into a mixer. The mixture is mixed for 45 minutes. The homogeneity of the mixture is checked visually.
3. The powder tablets are filled in powder bottles directly from the mixture.

A daily dose is twice of the single dosage shown above. This drink powder needs to be stirred in about 400 ml of water prior use.

### EXAMPLE 10

### The Effect of Russian Tarragon (Artemisia dracunculus L.) on the Plasma Creatine

Concentration with Creatine Monohydrate Administration

### Background:

It has previously been shown that the plasma concentration of creatine following supplementation is influenced by extracellular concentrations of insulin and glucose, the form in which creatine is administered, and also the creatine concentration in the muscle cells. The common practice of raising insulin levels to increase initial uptake into muscle, by means of high amounts of glucose and/or protein, involves a high caloric load which is not always desired by athletes. A standardized extract of Russian Tarragon (Artemisia dracunculus L.), which can be administered safely as an oral supplement, has been shown to have antihyperglycemic activity. This study examined whether the plasma concentration curve following administration of creatine monohydrate was affected by the co-administration of Russian Tarragon extract.

### Methods:

Eleven healthy male subjects (20.4+/−1.5 yrs, 180.0+/−7.2 cm) participated in the study. Each subject was assigned to ingest a single dose of 60 mg/kg bwt creatine monohydrate (Creapure™, AlzChem, Trostberg, Germany), preceded 15 minutes earlier by ingestion of 2×500 mg capsules of a standardized extract of Artemisia dracunculus L. (Finzelberg, Andernach, Germany) or placebo. Plasma creatine concentrations, determined over two hours following ingestion, were analyzed by repeated measures ANOVA.

### Results:

Russian Tarragon administration resulted in a significant reduction of plasma creatine levels at 60, 90 and 120 min, in comparison to placebo (FIG. 1), as well as a significant reduction in the area under the plasma concentration curve (AUC). The effect of Russian Tarragon is seen as comparable to that of glucose and protein. FIG. 1 shows mean (SD) plasma creatine concentration following administration of 60 mg/kg bwt creatine monohydrate, proceeded 15 minutes earlier by ingestion of 2×500 mg capsules of a standardized extract of Russian Tarragon (A) or placebo (B). The inset shows the mean differences (±SD) between treatments in the change from baseline at each time point.

### EXAMPLE 11

### The Effect of Russian Tarragon (Artemisia dracunculus L.) Combined with Creatine Monohydrate Administration on the Glucose Metabolism in Rats

### Animal Model for Glucose Challenge Test

A common way of testing for an effect of extracts or new chemical entities on blood glucose levels is the glucose challenge test (Verspohl, E. J.: Recommended testing in diabetes research. Planta Med 68 (7): 581-90, 2002) in which rats are given the extract, control, or a known antidiabetic substance with challenge an intraperitoneal (i.p.) dose of glucose. The extracts or extract PLUS creatine are given orally 30 min. before the glucose challenge. Blood samples are taken sublingual at time points 0, 15, 30, 60, and 120 minutes for the glucose challenge. This sampling scheme ensures to monitor both effects of extracts on the blood glucose levels after glucose challenge closely enough.

### Animals

Male non-fasted Wistar rats weighing 250-300 g were purchased from Harlan (Indianapolis, Ind., U.S.A). The non-fasted condition was chosen to account for a more physiological situation, but increases variability of blood glucose levels. Rats were housed in cages of 2 at 20±1° C. in a 12-h light/dark cycle. Tap water and food pellets were available ad libitum. Groups of 6 rats were randomly assigned to the 3 different treatment groups. All experiments were carried out in a quiet room between 9:00 a.m. and 2:00 p.m. All animals were housed and all experiments performed according to the policies and guidelines of the Institutional Animal Care and Use Committee (IACUC) of the University of Florida, Gainesville, U.S.A.

### Test Substances

All substances (extract 60 mg/kg or extract+creatine 1:5, in total amount 60 mg/kg, p.o.) were prepared by dissolving the various test materials in 5 ml deionized water with 0.5% propylene glycol to form a suspension. All solutions were prepared freshly on test days. All animals were brought to the testing room at least 30 minutes prior to testing and remained in the same room throughout the test. Animals were orally treated with control (vehicle), or the test substances, i.e. by oral administration via gavage. Glucose (Sigma-Aldrich) was dissolved (using sonication) in 0.9% saline solution in a concentration of 2 g/5 mL and given i.p. 30 minutes after the oral treatment.

### Blood Glucose Evaluation

Blood was drawn at the appropriate time points from the sublingual vein after a short halothane anesthesia and stored at 4° C. with addition of heparin. Samples were centrifuged at 8600 rpm for 10 minutes. The supernatant plasma was taken and analyzed using an autoanalyzer (Merck, Darmstadt). Analytical plasma controls and matrix blanks were used to guarantee accurate results within the specified limits.

### Statistics and Calculations

Both percent and AUC data were analyzed by one-way ANOVA and Student-Newman-Keuls Multiple Comparison Test using Graphpad 4.0 Software, San Diego, USA. The AUC (area under the curve) was calculated using the trapezoidal rule without extensions beyond the last time point measured.

### Results

FIG. 2 shows the higher effectiveness of a Russian Tarragon extract PLUS Creatine combination towards a more powerful glucose disposal from blood. Sole creatine monohydrate administration has got no effect on the glucose disposal compared to control whereas the combination of Russian tarragon extract and creatine resulted in a significant reduction of plasma glucose levels at 15, 30, and 60 min, in comparison to control or creatine alone (FIG. 2), as well as a significant reduction in the area under the plasma concentration curve (AUC). The effect of the combination of Russian Tarragon PLUS creatine regarding the shown increased glucose disposal from the blood is interpreted indirectly with a higher uptake of creatine into different tissues (e.g. muscle, nerve) due to the known coupled glucose and creatine uptake.

1. A product comprising
a) Russian Tarragon (Artemisia dracunculus), an extract of Russian Tarragon or a derivative thereof
b) a creatine and/or carnitine compound or derivative or precursor thereof.

2. The product of claim 1, wherein the Tarragon is selected from Russian Tarragon, and its varieties (e.g. var. inodora) and cultivars.

3. The product of claim 1, wherein a) is selected from extracts of Russian Tarragon with water or a mixtures of water with up to 20% by volume of C1 to C3 alcohol.

4. The product of claim 1 wherein the derivative is a constituent of Russian Tarragon selected from the group of carbohydrates, proteins, peptides, and polyphenols.

5. The product of claim 1, wherein the nutritive compound is selected from creatine, carnitine and mixtures thereof.

6. The product of claim 1, wherein the derivative of the creatine compound is selected from creatine hydrates, creatine salts, creatine monohydrate, creatine pyruvate, creatine ascorbate, creatine citrates, creatine malate, creatine tartrate, creatine orotate, creatine pyroglutamate, creatine ketoglutarate, creatine ethyl ester salts and/or precursors of creatine, such as arginine, glycine, methionine, guanidinoacetate and mixtures of thereof.

7. The product of claim 1, wherein the derivative of the creatine compound is selected from carnitine, carnitine salts, alkanoyl-carnitine, L-carnitine, L-carnitine hydrochloride, L-carnitine fumarate, L-carnitine L-tartrate, L-carnitine Magnesium citrate, Acetyl-L-carnitine hydrochloride, 3-lauroyl-L-carnitine hydrochloride, and carnitine creatinate or mixtures thereof.

8. The product of claim 1, wherein a) is in an amount of 100 to 25000 mg and b) is in an amount from 20 to 5000 mg.

9. A dietary supplement, food, beverage or pharmaceutical product comprising the product of claim 1.

10. The dietary supplement, food, beverage or pharmaceuticals product of claim 9, further comprising carbohydrates, like dextrose, maltose, maltodextrin and trehalose, formulation aids, like dissolution enhancer, binder and other auxiliaries, minerals, like Magnesium and Calcium, trace elements, like Vanadium, Chromium, Zinc, methylxanthines, like caffeine, theobromine and theophylline, free amino acids, like taurine, glutamine, citrulline, leucine, glycine, arginine, alanine, or salts of derivatives thereof, vitamins, like vitamin A, C, E, vitamin derivatives, herbs and botanical extracts with or without glucose-modifying effect, as well as lactic acid buffering agent, like (sodium) bicarbonate, citrates, phosphates, carnosine, beta-alanine, and mixtures thereof.

11. The dietary supplement, food, beverage or pharmaceuticals product of claim 9 in the form of a powder, of capsules, tablets, effervescent tablets, powdered beverages, bars, gels or drinks, pharmaceutical delivery systems.

12. Use of the product of claim 1 for enhancing athletical muscular performance, muscle strength, muscle mass, stamina, endurance, for improvement of physiological recovery and body-composition index (BCI), for avoidance of fatigue as well as for enhancing cognitive performance, neuronal tissue stress tolerance, for avoidance of dementia and neurodegeneration and metabolic diseases, like metabolic syndrome and diabetes type 2 in athletes, sport people or elderly people and ill patients.

13. Use of Russian tarragon (Artemisia dracunculus), an extract of Tarragon or a derivative thereof to increase uptake of creatine and/or carnitine substances.
